(19) United States
(12) Patent Application Publication
(10) Pub. No.: US 2010/0318047 A1
(43) Pub. Date: Dec. 16, 2010

(54) ABSORBENT, NONWOVEN MATERIAL EXHIBITING Z-DIRECTION DENSITY GRADIENT

(76) Inventors: Paul M. Ducker, Brunswick, GA (US); Steven S. Harlen, Jesup, GA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/485,293

(22) Filed: Jun. 16, 2009

(51) Int. Cl. A61F 13/51; B32B 37/14

(52) U.S. Cl. 604/365; 156/60

(57) ABSTRACT

The present invention relates to an absorbent material that can be used as an absorbent core in absorbent articles such as sanitary napkins, pantiliners, incontinence products, disposable diapers, etc. The material of the present invention is a nonwoven sheet, consisting of cellulosic fibers, optionally superabsorbent polymeric material, containing no binders, latexes, etc, relying on hydrogen bonding to produce the necessary structure. The material contains density gradients which direct fluid into the material and distribute it providing more effective fluid transport and efficient utilization of storage capacity. The material consists of two regions. In the first region, the material has a low-density stratum adjacent to one surface, overlaying at least one higher density stratum adjacent to the opposite surface of the sheet. These strata create a density gradient in the thickness direction (Z-direction) of the sheet. The second region consists of a fluid distribution structure that has a higher density than at least the lower density of the strata comprising the first region. The fluid distribution structure is in direct fluid communication with the adjacent strata in the first region, along their boundaries.

### TECHNICAL FIELD

The present invention relates generally to disposable absorbent materials that have structures suitable for fluid (liquid) intake, fluid storage and fluid distribution, and that exhibit a high level of absorbency, and more particularly to an improved, nonwoven material exhibiting a Z-direction density gradient, comprising a first, relatively low-density stratum, and a second, relatively high-density stratum, which are integrated and stabilized by hydrogen bonding, without resort to binder compositions or synthetic fibers, with the resultant material exhibiting desirably high tensile strength, and resistance to delamination. The absorbent material can be used in absorbent articles such as feminine hygiene products, incontinence products, and disposable diapers.

### BACKGROUND OF THE INVENTION

Disposable absorbent articles, such as diapers, feminine hygiene products, adult incontinence devices and the like have found widespread acceptance. To function efficiently, such absorbent articles must quickly absorb body liquids, distribute those liquids within and throughout the absorbent article, and be capable of retaining those body liquids when placed under loads.

While the design of individual absorbent articles varies depending on use, there are certain elements or components typically common to such articles. The typical absorbent article includes a liquid pervious top sheet or facing layer, which facing layer is designed to be in contact with a body surface. The facing layer is made of a material that allows for the unimpeded transfer of fluid from the body into the core of the article. The facing layer should not absorb fluid per se and, thus, should remain dry. The typical article further includes a liquid impervious back sheet or backing layer disposed on the outer surface of the article and which layer is designed to prevent leakage of fluid out of the article.

Disposed between the facing layer and backing layer is an absorbent member referred to in the art as an absorbent core. The function of the absorbent core is to absorb and retain body fluids entering the absorbent article through the facing layer. Because the origin of body fluids is localized, it is necessary to provide a means for distributing liquid throughout the dimensions of the absorbent core. This is typically accomplished either by providing a distribution member in the core and/or by altering the composition or structure of the absorbent core per se.

Since fluids can be presented more quickly to the absorbent product than the absorbent core can absorb, a distinct acquisition structure is frequently employed to capture liquid more quickly than it is added and retain it long enough for remainder of the core to absorb it out of the acquisition structure. This transfer of fluid to the core restores the capacity of the acquisition structure for subsequent fluid insults. It is advantageous for the core to absorb fluids more quickly as the acquisition structure can be smaller.

The absorbent core is frequently formulated of a cellulosic wood fiber matrix or pulp, which pulp is capable of absorbing large quantities of fluid. Fluid absorption and retention properties can be enhanced by disposing superabsorbent materials in amongst the fibers of the wood pulp. Superabsorbent polymeric materials (SAP) are well known in the art as substantially water-insoluble, absorbent polymeric compositions that are capable of absorbing large amounts of fluid in relation to their weight and forming hydrogels upon such absorption. Absorbent articles containing blends or mixtures of pulp and superabsorbents are known in the art. An example of this is taught in U.S. Pat. No. 3,670,731 (Harmon).

Fluid can be preferentially directed through an absorbent core by use of wettability and or density gradients existing in the core structure. Various embossed structures employ high-density wicking lines or other continuities that due to their density attract liquid from the adjacent core and then direct it away from the point of insult.

U.S. Pat. No. 4,781,710 (Megison et al) teaches an absorbent pad with low-density “tuft” regions, designed to quickly imbibe liquid in fluid communication with very dense fluid transport channels, which transport liquid away from the tuft regions, and enclosed by the transport channels are storage structures of a medium density. While this provides three differentiated structures to accomplish the various functions, the structures are coplanar, and do not direct fluid from the fluid receiving side of the core to the opposite face in the thickness direction.

Fluid can be transported away from the fluid receiving surface of an absorbent core by providing a density gradient in the thickness direction. U.S. Pat. No. 5,525,407 (Yang) provides a core with a density and wettability gradient in the thickness direction. The low-density layer on the fluid receiving side acquires liquid and directs it to higher density layers below. The fluid receiving side becomes desirably drier, and the denser layers below also spread the liquid in the in-plane directions utilizing portions of the core that are not directly beneath the point of fluid insult.

This technology produces the density gradients by forming various strata using a device called a transverse webber and differentiating the fiber types in each stratum using various blends of synthetic fibers or binder fibers and cellulosic fibers in different proportions. Ovens are used to activate the binding fibers followed by heated calenders. The synthetic fibers are expensive, they are not hydrophilic, and the ovens required to bond the material at high speeds are energy and capital intensive.

Absorbent materials made using commercial multibonded airlaid technology provides a method of manufacturing pre-formed structured absorbent cores to the absorbent article converting process. The use of pre-formed structured cores increases the efficiency of the converting operation by taking the complexity of forming or combining several core structures off of the converting machine. U.S. Pat. No. 6,420,626 (Erspamer, Buckeye) teaches a pre-formed unitary multibonded airlaid core with differentiated acquisition, fluid storage, and fluid transport strata with associated density gradients in the thickness direction. As in the previous example, this technology requires the use of expensive synthetic fibers and the requirement for large capital and energy-intensive bonding ovens to bond the material.

U.S. Pat. No. 5,866,242 (Tan) teaches an airlaid material, sometimes referred to as a hydrogen bonded airlaid, comprising cellulosic fibers, and optionally superabsorbent polymer that is bonded using heat and pressure to form hydrogen bonds. No synthetic binder fibers, heat fusible thermoplastics or other chemical binders are used. In commercial practice, a heated calender roll applies the pressure and heat required to form hydrogen bonds between the fibers. Compared to the muitibonded airlaid process previously cited, this bonding arrangement is much simpler to operate, has significantly less energy consumption, and requires much less capital expenditure than the bonding ovens used in the multibonded airlaid process. Additionally no synthetic binder fiber, fusible thermoplastic materials or chemical binders are required which would add cost to the material and are non-absorbent components. These binders can restrict the swelling of the SAP particles reducing their absorbency.

While the hydrogen bonded airlaid process and material as taught by Tan is very simple and cost-effective, the process does not produce strong density gradients in the thickness direction. Therefore, it would be desirable to have a hydrogen bonded airlaid material comprising only cellulose and optionally SAP with no chemical binders or synthetic bonding fibers, that has good sheet integrity, minimal fiber dusting, and a strong density gradient in the thickness direction.

### SUMMARY OF THE INVENTION

The present invention relates to an absorbent material that can be used as an absorbent core in absorbent articles such as sanitary napkins, pantiliners, incontinence products or disposable diapers. The material of the present invention is a nonwoven sheet, consisting of cellulosic fibers and SAP, containing no binders, latexes, etc, relying on hydrogen bonding to produce the necessary structure.

The material contains density gradients which direct fluid into the material and distribute it providing more effective fluid transport and efficient utilization of storage capacity. The material comprises two regions. In the first region, the material has a low-density stratum adjacent to one surface overlaying at least one higher density stratum. These strata create a density gradient in the thickness, Z-direction of the sheet. The second region includes a fluid distribution structure that has a higher density than at least the lower density one of the strata comprising the first region, and extends through the entire thickness of the sheet. The fluid distribution structure is in direct fluid communication with both the adjacent strata in the first region, along their boundaries.

In another aspect of the invention, the density ratio in the thickness direction is greater than 1.2:1. In another aspect of the invention, the types of cellulose comprising the various strata in the Z-direction can be differentiated. In another aspect of the present invention, the droplet absorption time differs between the two surfaces of the sheet with a droplet absorption time ratio>1.5:1. In another aspect of the present invention, the sheet has an effective containment mechanism for fibers in the low-density stratum in the first region to prevent dusting.

In another aspect of the present invention, the material has a tensile strength of at least 10 (N/50 mm) providing useful sheet integrity for use in converting processes to make absorbent articles. In another aspect of the present invention, liquid spreads in the X-Y direction in the various core structures to an extent according to their density. In a preferred embodiment, the material is produced using a hydrogen bonded airlaid process using heated calenders to provide the heat and pressure to effect the bonding.

In accordance with the illustrated embodiments, the present absorbent, nonwoven material comprises a first, relatively low-density stratum comprising a fibrous matrix of cellulosic fibrous material substantially free of synthetic fibers and binder compositions. The present material further comprises a second, relatively high-density stratum, juxtaposed to the first stratum in liquid-transferring relationship therewith. The second stratum comprises a fibrous matrix of cellulosic fibrous material substantially free of synthetic fibers and binder compositions.

The absorbent material further includes a liquid-distribution network extending substantially through the Z-direction of the absorbent material, which is formed from the first stratum and the second stratum. The liquid-distribution network thus extends substantially through the entire thickness of the material, and is provided so as to be laterally adjacent to at least some portions of the first, low-density stratum and the second, high-density stratum in liquid-transferring relationship therewith.

The present material further includes a cellulosic fiber tissue layer positioned on top of the low-density stratum, which tissue layer is bonded to the liquid-distribution network for integrating the absorbent material against delamination, and for inhibiting release of the fibrous material of the low-density stratum.

Notably, the present material is integrated and stabilized by hydrogen boding, formed by the application of heat and pressure, to thereby provide a nonwoven sheet with a machine direction (MD) tensile strength of at least 10 (Newtons/50 millimeter wide sample) and a vertical delamination strength of greater than 5N, with hydrogen boding serving to stabilize the density of the strata and the density gradient, and to stabilize the integrity of the liquid-distribution network and bonded tissue layer. Without being bound to any particular theory, it is believed that the hydrogen bonding between a portion of the fibers in the cellulosic fiber matrix are configured so as to hold it in a state of compression in the thickness direction. The resiliency of the remainder of the fibers pushes back against these compressive forces forming an equilibrium density. It is believed that this is a distinctive characteristic of hydrogen bonding formed by using a heated calender to form bonds on an airlaid cellulosic web since the web is compressed to form the bonds, but bonds are only formed between some of the fibers, and the rest rebound against those bonds according to their resiliency. It is believed that the beneficial effect of this equilibrium is that when external mechanical forces are applied to the structure such as compression, the tension and resiliency in the structure makes it tend to spring back to its equilibrium density. A distinctive feature of the material of the present invention is that the integrity of the densities of each of the various structures in the material of the present invention are in this way believed to be maintained as the material is formed into roll or festooned packages, converted into absorbent products, and manipulated during end use. In this way, it is believed the desirable functional aspects of these densities are likewise maintained.

In the preferred embodiment of the present invention, the absorbent material exhibits an apparent Z-direction density gradient greater than 1.1:1. In one embodiment, the liquid-distribution network comprises at least one longitudinally extending densified region. In an alternative embodiment, the liquid-distribution network comprises a land-and-sea densified region.

The liquid-distribution network of the present absorbent material comprises between about 5% and 50% of the surface area of the absorbent material, more preferably, between about 10% and 35% of the surface area of the absorbent material.

While it is within the purview of the present invention that the present absorbent material be formed only from cellulosic fibrous material, such as comminuted wood pulp, at least one of the strata of the absorbent material can include superabsorbent polymeric material. In such an embodiment, the absorbent material can be provided with a basis weight of about 100 to 2000 gsm (grams per square meter), and comprise between about 0% and 70%, by weight, of superabsorbent polymeric material. The low-density strata can be provided with a density in the range of 0.08 g/cc (grams per cubic centimeter), to about 0.30 g/cc, and the high-density strata provided with a density in the range of about 0.25 g/cc to 0.50 g/cc.

More preferably, when the present material includes superabsorbent polymeric material, the absorbent material can be provided with a basis weight of about 150 to 1000 gsm, and comprises about 10% and 55%, by weight, of the superabsorbent polymeric material. The low-density strata can be provided with a density in the range of about 0.10 g/cc to 0.17 g/cc, and the high-density stratum provided with a density in the range of about 0.25 g/cc to 0.40 g/cc.

In the preferred embodiment, the high-density stratum of the present absorbent material includes another cellulosic tissue layer at the lower surface thereof, with the cellulosic tissue layers being bonded together along the liquid-distribution network of the material.

It is within the purview of the present invention that the fibrous material of the low-density stratum is different than the fibrous material of the high-density stratum.

In accordance with the disclosed testing protocols, the absorbent material in accordance with the present invention has a droplet absorption time ratio of greater than or equal to 1.5:1.

A method of making the present absorbent, nonwoven material comprises the steps of providing a cellulosic tissue layer, and depositing cellulosic material on the tissue layer. The cellulosic material is compacted to form a fibrous matrix of a first, relatively low-density stratum.

The present method further includes providing a second, relatively high-density stratum with hydrogen bonding formed by applying heat and pressure providing a stable density, with the second stratum comprising another fibrous matrix, and with the second stratum provided on the first stratum.

The method further comprises compacting the first and second strata by applying heat and pressure in a defined pattern, to form an absorbent material with a Z-direction density gradient. Formation includes forming a liquid-distribution network extending through the entire thickness of the material, which network is laterally adjacent to at least one portion of the low-density and high-density strata. The cellulosic tissue layer of the material is bonded to the liquid-distribution network for integrating the absorbent material against delamination, and for inhibiting release of the fibrous material of the first, low-density stratum. In accordance with illustrated embodiments, the liquid-distribution of the material is formed with at least one longitudinally extending densified region, or alternatively, is formed to comprise a land-and-sea densified region. As noted, at least one of the fibrous matrices of the present absorbent material can be provided in the form of a blend of cellulosic fibrous material and superabsorbent polymeric material.

In the preferred method of making the present absorbent material, the step of providing high-density stratum includes forming and compacting the high-density stratum separately from the low-density stratum, and thereafter positioning the high-density stratum on the low-density stratum.

In the most preferred form, the absorbent material is formed using an airlaid formation apparatus, comprising a single formation section, and a single bonding calender positioned downstream of the single formation section.

Other features and advantages of the present invention will become readily apparent from the following detailed description, the accompanying drawings, and the appended claims.

### BRIEF DESCRIPTION OF THE DRAWINGS

FIG. 1 is a diagrammatic, cross-sectional view of an absorbent, nonwoven material exhibiting a Z-direction gradient embodying the principals of the present invention; and

FIG. 2 is a diagrammatic, cross-sectional view of an alternative embodiment of the present absorbent, nonwoven material.

### DETAILED DESCRIPTION

While the present invention is susceptible of embodiment in various forms, there is shown in the drawings and will hereinafter be described presently preferred embodiments of the invention, with the understanding that the present disclosure is to be considered as an exemplification of the invention, and is not intended to limit the invention to the specific embodiments illustrated.

The present invention provides a novel absorbent nonwoven material suitable for use in absorbent articles that comprises cellulosic fibers and optionally superabsorbent polymer (SAP) with no synthetic fibers or chemical bonding agents. The density structure of the material is maintained through hydrogen bonding and provides improved absorbency characteristics by directing the flow of liquid through the structure along the density gradients. Included in the structure is a strong density gradient in the thickness direction over at least a portion of the surface.

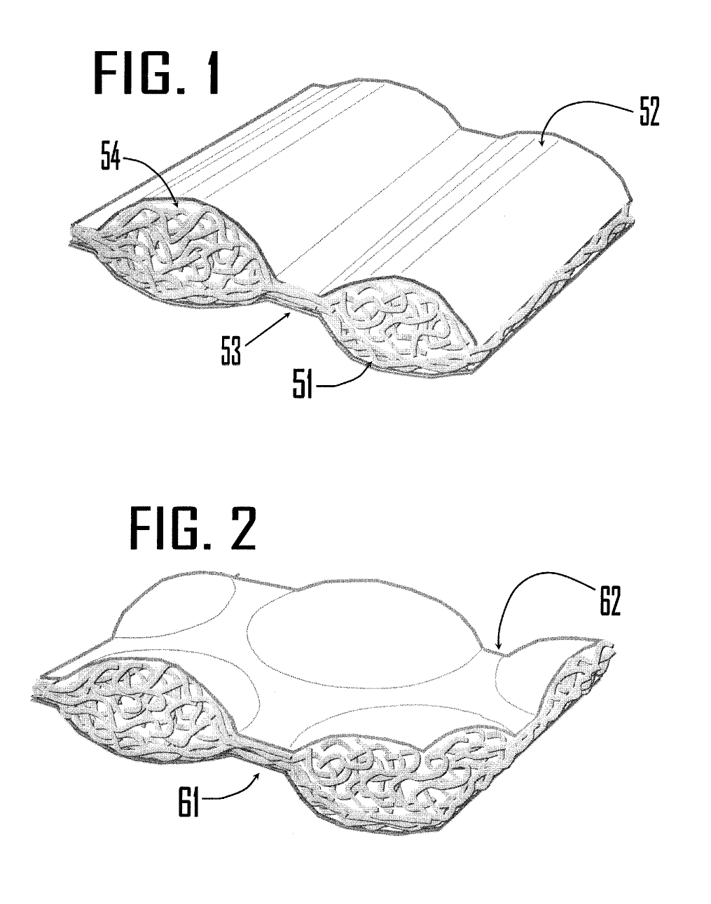

Referring to FIG. 1, cellulosic fibers (51, 53, and 54) are enclosed between two sheets of cellulosic tissue on top (52) and bottom (not shown). The material is divided into two regions, which in this particularly preferred embodiment alternate in a parallel pattern, including at least one longitudinally extending densified region, which preferably extends in the machine direction (MD) of the material, Any number of patterns may be suitable, however, depending on the desired physical and absorbent characteristics of the sheet. The first region comprises a stratum of relatively low-density fibers (54) adjacent to one face of the sheet overlaying at least one higher density stratum of more heavily bonded fibers (51), forming a density gradient in the thickness direction. The low-density fibers (54), many of which are largely unbonded, are contained by the top tissue (52) to prevent dusting during handling. In the second region, cellulosic fibers bonded to a relatively high density extend through the entire thickness of the material, and are in fluid communication both strata (51, 54) of the first region at the boundary between the two regions. The top tissue (52) is strongly bonded to the fibers 51 in the second region providing effective delamination strength for the tissue layer. Any of the fiber structures 51, 53, or 54 can optionally contain superabsorbent polymeric material SAP granules (not shown). In this preferred embodiment, the high-density bonded second region extends in the machine direction of the sheet, providing a strong preferential longitudinal wicking of liquid. In alternative embodiments, the second region can be in a series of discrete disconnected shapes, with the second region providing little wicking in the in-plane directions or in an alternative embodiment, shaped to have continuities in both the length and width direction providing in-plane wicking in both directions. The second region can comprise between 1% to 90% of the surface, but is desirably between 5% and 40% of the surface area, and more desirably between 15% and 35% of the surface.

Cellulosic fibers that can be used in the process of the present invention are well known in the art and include wood pulp, cotton, flax, and peat moss. Comminuted wood pulp is usually preferred. Pulps can be obtained from mechanical or chemi-mechanical, sulfite, kraft, pulping reject materials, organic solvent pulps, etc. Both softwood and hardwood species are useful. Softwood pulps are preferred. The pulp is most desirably provided in a package that can be processed by the airlaid equipment used to create the material of the present invention.

In another aspect of the present invention, the cellulosic fibers used to create the upper and lower strata that form the gradient can be differentiated in order to enhance the effectiveness of the gradient. One example of this would be to use cellulosic fibers, at least some of which have been made by a process that includes the step of treating a liquid suspension of pulp at a temperature of from about 15° C. to about 60° C. with an aqueous alkali metal salt solution having an alkali metal salt concentration of from about 2 weight percent to about 20 weight percent of the absorbent material.

Superabsorbent polymers (SAP) are well known in the art. As used herein, the term “superabsorbent polymeric material” means a substantially water-insoluble polymeric material capable of absorbing large quantities of fluid in relation to their weight. The superabsorbent material may be in the form of particulates, fibers, flakes, granules, or aggregates. Exemplary and preferred superabsorbent materials include salts of cross-linked polyacrylic acid such as sodium polyacrylate. Superabsorbent materials are commercially available (e.g., from Stockhausen GmbH, Krefeld, Germany). A wide range of types of are used in various disposable absorbent products; the appropriate grade depends very much on the required absorbency characteristics of the end use article. Those skilled in the art can select the optimal grade for the particular end use design.

The absorbent material of the present invention can incorporate an optional carrier tissue, and another optional tissue layer on top of the web. Suitable tissue materials for use are well known to those of ordinary skill in the art. Preferably, such tissue is made of bleached wood pulp and has an air permeability of about 273-300 CFM. Tissue for use in air-laying absorbent materials is commercially available (e.g. From Cellu Tissue in East Hartford, Conn.).

The absorbent material of the present invention can be configured in a uniform manner or can be configured with many strata of differing compositions of cellulose and/or superabsorbent. Those skilled in the art of making airlaid absorbent materials can design the optimal configuration for any given end-use product application. A preferred material may be configured with a top tissue and a carrier tissue, and have a substantially uniform homogeneous mix of cellulosic fibers and SAP.

A preferred method of producing the material of the present invention is to use the hydrogen bonded airlaid process. For purposes of this patent, a hydrogen bonded airlaid material is any nonwoven comprising cellulosic fibers and optionally superabsorbent polymer that is formed by suspending individualized fibers in an air-stream and depositing them in an undensified web by sending them through the forming heads of an airlaid web forming machine. Then hydrogen bonds are formed in the material.

Examples of several airlaid web forming machines are described in detail in U.S. Pat. No. 5,527,171 (Soerensen), hereby incorporated by reference. The forming heads may include rotating or agitated drums which serve to maintain fiber separation until the fibers are pulled by a vacuum onto a foraminous condensing drum or foraminous forming conveyor (or forming wire). Where multiple defined strata are desired, such as those having different compositions or densities, separate forming heads may be used to sequentially form each stratum on top of the stratum previously formed. As the fibers are airlaid the resulting structure is densified and the fibers are bonded together. by applying heat and pressure to the web to form hydrogen bonds, increasing the density and strength of the material compared to its undensified state. No chemical or thermoplastic binder materials are used. A preferred method of applying heat and pressure is to use a heated calender roll. Optionally, a cellulosic carrier tissue and a cellulosic top tissue can be used, which will bond and integrate into the web under this process.

In a preferred method to form the material of the present invention, cellulosic fibers are defiberized in a hammermill and deposited onto a moving forming wire covered with a carrier tissue. Superabsorbent polymer is optionally metered and blended in the forming head.

This web is then densified using a heated calender with a flat surface at a preferred temperature of >100 C but more preferably in the range of 150-200 C and a pressure necessary to obtain the desired density of the bottom stratum.

Additional cellulosic fibers and optionally SAP are deposited by a forming head onto a moving wire covered with a carrier tissue. The web thus formed is then combined with the first web at an embossed calender station. In a preferred embodiment, the embossed calender station has a temperature >100° C. and preferably in the range of 150°-200° C. and a pressure necessary to obtain the desired density and bonding in the embossed regions of the material.

In accordance with the preferred method of practicing the present invention, a cellulosic tissue layer is provided, on which cellulosic material is deposited, and compacted to form a fibrous matrix of a first, relatively low-density stratum. A second, relatively high-density stratum is thereafter provided on the first stratum, which second stratum comprises another fibrous matrix. Hydrogen bonding formed by applying heat and pressure provide a stable density.

The first and second strata are compacted by applying heat and pressure in a defined pattern, to form an absorbent material with a Z-direction density gradient, including forming a liquid-distribution network extending through the entire thickness of the material. The liquid-distribution network is provided laterally adjacent to at least one portion of the low-density and high-density strata, with the cellulosic tissue layer bonded to the liquid-distribution network for integrating the absorbent material against delamination, and for inhibiting release of the fibrous material of the first, low-density stratum.

Preferably, the step of providing the high-density stratum includes forming and compacting the high-density stratum separately from the low-density stratum, and thereafter positioning the high-density stratum on the low-density stratum. Since a typical airlaid apparatus has a single forming section, and it is desirable to avoid additional capital to reconfigure the machine, one method of forming the two strata is to form them one alongside the other on the same forming section, slit them apart into two webs, and then combine them after calendering one web first. A calender with an embossed pattern on one half of the roll, along with appropriate web routing can be used to produce the desired configuration with a single calender. Another approach using a single forming section is to form part of the web and then remove it from the wire at it's midpoint and then form the second stratum on the remainder of the wire. While there are many approaches, it is preferable to use an airlaid formation apparatus, comprising a single formation section, and a single bonding calender positioned downstream of the single formation section.

The embossed, densified regions can be provided in the form of a “land and sea” pattern of highly bonded areas surrounded by essentially non-bonded areas, or alternatively, more sophisticated patterns comprising intermediate densities or gradients. A particularly advantageous pattern comprises parallel lines of high density, oriented in the longitudinal direction, (Illustrated in FIG. 1) which preferentially distributes liquid along the orientation of the lines, which can be useful to avoid side leakage in an absorbent product. Useful patterns that maximize the intake function have land-sea patterns less than 50% bonded, and more preferably less than 30% bonded, but desirably more than 10% bonded, and generally can yield materials with useful mechanical integrity.

The material of the present invention has a basis weight of 100-2000 gsm, and more desirably in the range of 150-1000 gsm. The SAP content can range from 0-70%, but is more desirably in the range of 10-55% The density of the low-density stratum, as indicated by the basis weight and density procedure described below can range from 0.08 g/cc to 0.30 g/cc, with a more desirable range being from 0.10 g/cc to 0.17 g/cc. The apparent density of the high density stratum as indicated by the procedure below can range from 0.25 g/cc to 0.50 g/cc, with a more desirable range of 0.25-0.40 g/cc. The thickness direction density gradient in the first region, defined by the ratio of the densities of these two strata, is preferably in the range of 1.2-5.0, and even more preferably in the range of 1.5 to 2.5. The density of the material in the second region, as measured by the small area density test procedure described below, is ideally in the range of 0.30 g/cc to 1.2 g/cc, and more preferably 0.60 g/cc to 0.95 g/cc.

The embossed second region desirably comprises on average 5-50% of the material surface, more desirably comprising on average 10-35% of the surface area and more desirably comprises a repeating pattern, although this is not a requirement for the material of the present invention. The pattern of embossments can have no lateral continuity (as in discreet dots, or other geometric shapes), can have lateral continuity in one direction, (such as parallel lines, non-intersecting zigzags, etc) or have lateral continuity in both directions ( outlines of diamonds, squares, hexagons, etc). FIG. 1 illustrates a second region comprising pattern of higher-density parallel lines, while FIG. 2 illustrates a material with a first region comprising lower-density circles surrounded by the higher density second region. The pattern of embossment can also be designed to correspond to the shape of the core in the finished absorbent article, being used in a phased manner to provide particular properties such as mechanical strength or fluid transport in particular locations in the finished product where they are most suitable.

In one attempt by the applicants to produce a hydrogen bonded material comprising only pulp and SAP with a density gradient in the thickness direction, an airlaid mat of fluff and SAP was formed and then was run through a calender nip, with only the bottom roll of the nip heated. A mild density gradient was thus formed under process conditions that did not appreciably bond the fibers of the lower density top stratum. The unbonded fibers on this surface created unacceptable clouds of fiber dust when the web was handled. A top tissue layer to contain the dust would not bond to the web unless higher pressures were applied that caused the density gradient to disappear. The material of the present invention solves this problem in a very practical manner by having strong bonds available in the dense second region attach a top tissue layer in a robust manner to the web which then provides very effective containment of the fiber in the low-density stratum of the first region. The density of the second region required to form these effective bonds is dependent on the percent of the surface area that the second region comprises.

The prior art literature contains references to cores with density gradients in the thickness direction exhibiting greater wicking in the X and Y directions (in-plane) on the side of the sheet that has the higher density than the side of the sheet with the lower density. An example of this is in U.S. Pat. No. 5,525,407 (Yang). The material of the present invention clearly exhibits similar behavior, with liquid added at one location being spread furthest in the high density wicking lines of the second region, followed by the higher density stratum in the first region, with the least amount of spreading observed in the low-density stratum of the first region. As indicated in the prior art literature, this property is useful in hiding unsightly stains in various sanitary napkin and pantiliner products.

This behavior was quantified for the material of Example 1, by cutting a 12-inch×1-inch strip of material, suspending it vertically with one end in a pan of 0.9% saline solution and measuring the vertical wicking height after 30-minutes. The wicking height was the average of the highest vertical extent of liquid and the lowest vertical extent of liquid seen in the one-inch strip, within each density region.

The average vertical wicking height for the lower density stratum in the first region was 11.7 cm. The average vertical wicking height for the higher density stratum in the first region was 16.4 cm. (as viewed on the other side of the sample strip) The average vertical wicking height for the parallel embossed lines in the second region was 17.9 cm. The difference in vertical wicking height between the two strata in the first region as viewed on opposing sides of the sample strip is believed to be the effect of the density gradient in the sheet thickness direction in this region.

An unexpected additional benefit of the density gradient was observed when small amounts of viscous liquid were applied to the material of the present invention, according to the droplet absorption time test procedure outlined below. Since the small measured amounts of liquid added do not fully saturate the material, there is a strong component of fluid flow in the thickness direction, along the axis of the density gradient of the present invention, quantifying its effects on the rate of fluid flow through the thickness of the material. It was found that measured droplets applied to one side of the sheet were absorbed more slowly than similar amounts of liquid applied to the other side of the sheet. Without being bound by any particular theory, it is believed that the density gradient in the thickness direction is drawing the liquid into the sheet in a preferred direction, with the tendency to enhance flow rate from the low-density stratum to the higher-density stratum. Additionally, it was found that materials with similar basis weight and features as the material of the present invention, but without the density gradient in the thickness direction, exhibited none of the asymmetry of absorption time, one side versus the other, and the speed of absorption was slower than that of the material of the present invention in the direction believed to be enhanced by the density gradient in the thickness direction.

Since absorbency rate is critical to avoiding leakage, this property of the material of the present invention can be very useful in making improved absorbent cores for absorbent articles, particularly those that handle more viscous liquids such as sanitary napkins.

Test Procedures:

Basis weight and density: A 300 mm×200 mm hand sheet of sample material is cut using an Atom model SE 20C die press from Associated Pacific Company of Camarillo Calif. using an appropriately sized cutting die. The sample is weighed on a lab balance readable to 0.001 g. The sample is then placed in an Emveco Microgauge, with a foot pressure of 0.7 psi. Caliper is measured in 6 locations about the sheet and the average is taken using the average function.

The basis weight in grams per square meter is divided by the caliper in millimeters and this is divided by 1000 to yield density in units of grams per cubic centimeter.

Z—Direction Density Ratio:

A sample of web is taken immediately after the smooth calender, but prior to any additional fiber being added, with a stable process operating at constant speed, which is the intended operation for a commercial airlaid process. This material becomes the higher density stratum in the first region, when the process is completed. Taking the sample at this point allows this stratum to be measured without having to separate it from the remainder of the material. 200 mm×300 mm samples (quantity: 10) are taken from this material and the basis weight and density test listed above is performed. The average value is representative of the structure.

With the process still operating at the same constant speed and stable state, a sample of the finished material is taken. 200 mm×300 mm samples (quantity: 10) are taken and again the basis weight and density test is performed on these samples and average values recorded.

Calculations:

Average measurements were taken for the basis weight and caliper of the entire sheet, and for the basis weight and caliper of the higher density stratum, taken directly after it is bonded by the smooth calender in the process but before any additional fiber is added.

The following calculations are then done to calculate the basis weight and caliper of the lower density stratum, which cannot be separated to be measured by itself:

average basis weight (entire sheet)−average basis weight (high density stratum)=basis weight (low density stratum)

average caliper (entire sheet)−average caliper (high density stratum)=caliper (low density stratum)

The density of the high density stratum and the low density stratum are then calculated:

density (high density stratum g/cc)=basis weight (high density stratum, gsm)/caliper (high density stratum, mm)/1000

density (low density stratum g/cc)=basis weight (low density stratum, gsm)/caliper (low density stratum, mm)/1000

Finally, the density ratio between the high density stratum and the low density stratum is calculated.

density (high density stratum, g/cc)/density (low density stratum, g/cc)=Z-direction density ratio

Small Area Density:

The small dense areas of the second region are generally too small to be measured using the Emveco Microgauge. Therefore, an MHC brand mechanical dial indicator gauge on a magnetic base or the equivalent with a dial indicator readable to 0.001 inch and a 0.09-inch ball end probe was used. When the dial indicator was allowed to push downwards vertically on the weigh pan of a Sartorius lab balance, 73 g was the reading. The dial indicator is placed on a flat metal surface and the magnetic base is engaged. The mounting bracketry is then adjusted for the dial indicator to be oriented vertically with the ball in contact with the smooth metal surface. The bezel is then rotated to yield a zero reading. Caliper readings are taken by lifting the probe and placing a sample beneath it so that the probe rests on the dense portion of the second region. The caliper of is read directly in millimeters.

Sampling for Small Area Density Testing:

A 300 mm×200 mm hand sheet of sample material is cut using an Atom model SE 20C die press from Associated Pacific Company of Camarillo Calif. using an appropriately sized cutting die. The sample is weighed on an electronic lab balance (Sartorius, or the equivalent) readable to 0.001 g. The weight is divided by the area to yield a calculated average basis weight in grams per square meter. Then 5 caliper readings are taken using the dial indicator gauge according to the above procedure, taken from equivalent locations in the second region. (in the case of the example materials, the second region consists of parallel lines embossed by a process intended to yield a constant caliper. The caliper readings were taken along the centerline of these parallel lines) The basis weight for the sheet in grams per square meter is divided by the average of the 5 caliper measurements in millimeters and divided by 1000 to yield the average density of the readings.

Tensile:

A 240 mm×50 mm sample is cut using an Atom Model SE 20C die press from Associated Pacific Company of Camarillo, Calif. and an appropriately sized cutting die. A tensile test is then done on the strip using a Zwick Model Z005 tensile tester from Zwick/Roell in Ulm, Germany, or the equivalent. The test starts at a 200 mm jaw separation. The sample is placed in the jaws and the force is zeroed. The tester program then applies 2N pre-load to the strip at a rate of 100 mm/min and then proceeds to pull the sample at a rate of 100 mm/minute until failure, recording the maximum force in Newtons per the 50 mm wide test strip.

Droplet Absorption Time:

This test measures the time required for the capillary action of the core to draw a measured dose of a viscous liquid into it. This test uses a test fluid consisting of 0.9% saline solution (available as a prepared solution from Lab Chem, of Pittsburg, Pa., Catalog No. 07933), with enough Sodium Carboxymethylcellulose (Hercules Chemical, Type 7a) fully dissolved to yield a homogeneous solution with a stable viscosity of 30±2 centipoise, measured with a Brookfield Syncro-Lectric viscometer at 75 degrees C.

A 300 mm×200 mm sample of material is cut using an Atom model SE 20C die press from Associated Pacific Company of Camarillo Calif. using an appropriately sized cutting die. The sample is weighed on a lab balance readable to 0.001 g. The weight is divided by the area of the sample in square meters to yield the basis weight in grams per square meter. The dose of test fluid in cubic centimeters required is the basis weight of the sheet multiplied by a factor of 0.00044. The calculated dose of test fluid is drawn up into a 1 cc tuberculin syringe without needle (available from BD Medical of Franklin Lakes, N.J. Catalog No 309602) with graduations readable to 0.01 cc. The end of the syringe is held one droplet diameter above the material sample oriented vertically and the dose is dispensed in about 1 second (to avoid squirting and unduly spreading the droplet over a larger surface). At the beginning of the dosing, a stopwatch is started and the time required for the droplet to completely absorb into the sheet is recorded. The end point is when the last specular liquid surface disappears into the material.

Sampling for the Droplet Absorption Time Test:

Ten (10) droplets are placed in separate un-wetted locations on one side of the material according to the procedure above and the average and standard deviation of the absorption time is recorded. In the case of materials in which the first and second region dimensions are large relative to the droplets, the droplets should be placed in the first region, which contains the density gradient in the thickness direction. The sample is then turned over and ten (10) droplets are placed on the other side, in un-wetted locations, again according to the procedure above, and likewise the average and standard deviation of the absorption time is recorded. The average absorption time of the less dense side is then divided by the average absorption time of the more dense side. This is the droplet absorption time ratio.

### EXAMPLE 1

A nonwoven sheet material was made according to the present invention. The material comprised cellulosic fiber (Rayfloc J-LDE) from Rayonier, Jesup, Ga.), SAP (SA65s from Sumitomo Seika in Singapore) and 17 gsm 3995 tissue, (Cellu tissue, East Hartford, Conn). The first stratum formed had a total basis weight of 150 gsm, comprising cellulosic fiber and 15% SAP and included a layer of 17 gsm carrier tissue. Except for the carrier, the stratum was a homogeneous mix of SAP and cellulose, and was densified using a calender with a smooth surface on one roll and a linen pattern on the other heated to 170° C. at a sufficient pressure to yield a density of 0.31 g/cc. To this was added an additional stratum of material, with a total basis weight of 150 gsm, again comprising cellulosic fiber and SAP and including a layer of 17 gsm tissue, this time on the top. Except for the top tissue, the second stratum was likewise a homogeneous mix of SAP and Cellulose. This web was run through an embossed calender, heated to 170° C., comprising a pattern of parallel circumferential raised ridges of sinusoidal section, using the engraving pattern designated as 57RE80 from BF Perkins, of Rochester, N.Y. This creates a pattern of parallel embossed lines in the material running in the machine direction. The roll pressure was sufficient to produce a small area density measurement along the centerline of the embossed lines of 0.75 g/cc. The material had an overall basis weight of 300 gsm, an overall SAP content of around 15%, and an overall density of 0.22 g/cc.

### EXAMPLE 2

A material was made according to the present invention. The material comprised the same raw materials as Example 1. The first stratum had a total basis weight of 116 gsm, comprising cellulosic fiber and 30% SAP by weight and included a layer of 17 gsm carrier tissue. Except for the carrier, the stratum was a homogeneous mix of SAP and Cellulose, and was densified using a calender with a smooth surface on one roll and a linen pattern on the other heated to 170° C. at a sufficient pressure to yield a density of 0.28 g/cc. To this was added an additional stratum of material with a total basis weight of 111 gsm, again comprising cellulosic fiber and 25% SAP by weight and including a layer of 17 gsm tissue, this time on the top. Except for the top tissue, the second stratum was likewise a homogeneous mix of SAP and Cellulose and the web was run through an embossed calender, with the same embossing pattern as Example 1, heated to 170 C, with a sufficient force to produce a small area density measurement along the centerline of the embossed lines of 0.86 g/cc. The material had an overall basis weight of 227 gsm, a SAP content of around 30%, and an overall density of 0.20 g/cc.

### EXAMPLE 3

A material was made according to the present invention. The material comprised the same raw materials as Example 1. The first stratum had a total basis weight of 106 gsm, comprising cellulosic fiber and 25% SAP by weight and included a layer of 17 gsm carrier tissue. Except for the carrier, the stratum was a homogeneous mix of SAP and cellulose, and was densified using a calender with a smooth surface on one roll and a linen pattern on the other heated to 170° C. at a sufficient pressure to yield a density of 0.28 g/cc. To this was added an additional stratum of material with a total basis weight of 107 gsm, again comprising cellulosic fiber and 25% SAP by weight and including a layer of 17 gsm tissue, this time on the top. Except for the top tissue, the second stratum was likewise a homogeneous mix of SAP and cellulose. The web was run through an embossed calender, with the same embossing pattern as Example 1, heated to 170 C, with a sufficient force to produce a small area density measurement along the centerline of the embossed lines of 0.81 g/cc. The material had an overall basis weight of 213 gsm, a SAP content of around 25%, and an overall density of 0.17 g/cc.

### EXAMPLE 4

A material was made according to the present invention. The material comprised the same raw materials as Example 1. The first stratum had a total basis weight of 91 gsm, comprising cellulosic fiber and 10% SAP by weight and included a layer of 17 gsm carrier tissue. Except for the carrier, the stratum was a homogeneous mix of SAP and cellulose, and was densified using a calender with a smooth surface on one roll and a linen pattern on the other heated to 170° C. at a sufficient pressure to yield a density of 0.29 g/cc. To this was added an additional stratum of material with a total basis weight of 110 gsm, again comprising cellulosic fiber and SAP and including a layer of 17 gsm tissue, this time on the top. Except for the top tissue, the second stratum was likewise a homogeneous mix of SAP and cellulose. This web was run through an embossed calender, with the same embossing pattern as Example 1, heated to 170° C., with a sufficient force to produce a small area density measurement along the centerline of the embossed lines of 0.82 g/cc. The material had an overall basis weight of 201 gsm, a SAP content of around 20%, and an overall density of 0.19 g/cc.

### EXAMPLE 5

A material was made according to the present invention. The material comprised the same raw materials as Example 1. The first stratum had a total basis weight of 91 gsm, comprising cellulosic fiber and 10% SAP by weight and included a layer of 17 gsm carrier tissue. Except for the carrier, the stratum was a homogeneous mix of SAP and cellulose, and was densified using a calender with a smooth surface on one roll and a linen pattern on the other heated to 170° C. at a sufficient pressure to yield a density of 0.29 g/cc. To this was added an additional stratum of material with a total basis weight of 87 gsm, again comprising cellulosic fiber and SAP and including a layer of 17 gsm tissue, this time on the top. Except for the top tissue, the second stratum was likewise a homogeneous mix of SAP and cellulose. This web was run through an embossed calender, with the same embossing pattern as Example 1, heated to 170° C., with a sufficient force to produce a small area density measurement along the centerline of the embossed lines of 0.91 g/cc. The material had an overall basis weight of 178 gsm, a SAP content of around 20%, and an overall density of 0.19 g/cc.

Control 1:

A material was made to serve as a control to Example 3. It was intended to have a similar basis weight, SAP percentage, used similar raw materials, and had tissue on the top and bottom. The single stratum comprised cellulosic fiber and SAP and included a layer of 17 gsm carrier tissue on both the top and bottom. Except for the carrier layers, the stratum was a homogeneous mix of SAP and cellulose, and was densified using a calender with a smooth surface on one roll and a linen pattern on the other heated to 170° C. at a sufficient pressure to yield a density of 0.32 g/cc, giving it density features similar to the higher density stratum in the first region. The overall material had a basis weight of 217 gsm and a SAP content of around 25%.

Control 2:

A material was made to serve as an alternative control to Example 3. It was intended to have similar basis weight, SAP percentage, used similar raw materials and had tissue on the top and bottom. The entire material, however, was formed as one stratum and was bonded using the embossed calender, creating the features of the second region, but did not produce a density gradient in the first region. The single stratum comprised cellulosic fiber and SAP and included a layer of 17 gsm carrier tissue on both the top and bottom. Except for the carrier layers, the stratum was a homogeneous mix of SAP and cellulose, and was densified using a calender with a smooth surface on one roll and a linen pattern on the other heated to 170 C at a sufficient pressure to yield a density of 0.15 g/cc. The overall material had a basis weight of 225 gsm and a SAP content of around 25%.

Basis Weight and Caliper measurements were taken on the 5 example materials, according to the sampling and procedures explained above. Table 1 contains the measured and calculated densities obtained for the various samples and the resulting apparent density ratio in the thickness (Z-direction).

TABLE 1

| | | | Calculated | |
|---|---|---|---|---|
| | | Average | Density of | Calculated |
| | Average | Density of | Low-Density | Z-direction |
| | Density | High Density | Stratum | Density |
| | (g/cc) | Stratum (g/cc) | (g/cc) | Ratio |
| Example 1 | 0.22 | 0.31 | 0.17 | 1.8 |
| Example 2 | 0.20 | 0.28 | 0.16 | 1.7 |
| Example 3 | 0.17 | 0.28 | 0.12 | 2.4 |
| Example 4 | 0.19 | 0.29 | 0.15 | 1.9 |
| Example 5 | 0.19 | 0.29 | 0.14 | 2.1 |

It can be seen from the values above that the material of the present invention contains a substantial density gradient in the thickness direction, as quantified by the calculated Z-direction density ratio values.

The small area density was measured for the 5 example materials according to the method and sampling described above, measured along the center of the dense lines in the second region. Table 2 reports these average values obtained for Examples 1-5

TABLE 2

| | Small Area |
|---|---|
| | Average Density |
| | (Center of Second |
| | Region) g/cc |
| Example 1 | 0.75 |
| Example 2 | 0.86 |
| Example 3 | 0.81 |
| Example 4 | 0.82 |
| Example 5 | 0.91 |

Despite the differences in apparatus, the data suggests that the density in the second region is higher than the density in the first region.

Tensile measurements were taken for the various examples of the material of the present invention. The average tensile values are reported in Table 3 below:

TABLE 3

| | Average |
|---|---|
| | Tensile |
| | (N/50 mm) |
| Example 1 | 38 |
| Example 2 | 17 |
| Example 3 | 18 |
| Example 4 | 23 |
| Example 5 | 20 |

These data suggest that the hydrogen bonding in the materials of the present invention provides robust tensile strength.

Examples of the present invention and control materials were tested according to the droplet absorption time test procedure and sampling described above. These values are reported in Table 4 below:

TABLE 4

Droplet Absorption
Time (sec)

| Examples | Average | Average | |
|---|---|---|---|
| of the | Value, Low- | Value Low- | |
| Present | Density Side | Density Side | |
| Invention | Up | Down | Ratio |
| Example 1 | 3.8 | 9.1 | 2.4 |
| Example 2 | 3.6 | 7.1 | 2.0 |
| Example 3 | 3.5 | 7.9 | 2.3 |
| Example 4 | 3.2 | 7.5 | 2.4 |
| Example 5 | 3.3 | 8.9 | 2.7 |
| Control | | Top Side | |
| Samples | Top Side Up | Down | Ratio |
| Control 1 | 6.7 | 6.5 | .97 |
| Control 2 | 5.2 | 5.8 | 1.1 |

Examples 1-5 all exhibit a substantial difference in average droplet absorption times when comparing adding the liquid to the low-density side as opposed to adding the liquid to the high-density side.

Control 1 has a similar basis weight and SAP content as Example 3, but it has a generally uniform density throughout its thickness that is formed in a similar manner and looks like the higher density stratum in the first region of Example 3. As would be expected, the droplet absorption time is very similar for liquid added on one side of the sheet compared to being added on the other side. The absorption time for liquid added to Example 3 on the low-density side is faster than for this control, which does not have a gradient in the thickness direction.

Control 2 has a similar basis weight and SAP content as Example 3, but it is entirely bonded using the embossed calender. While the density and shape of the embossed second region is similar to that of the second region in Example 3, the density of the material in the first region looks more like the low-density stratum of the first region in Example 3. There is no density gradient in the thickness direction. As would be expected, the droplet absorption time is very similar for liquid added on one side of the sheet as compared to being added on the other, even though the surface relief resulting from the embossing is higher with the “top side up” than with the “top side down”. The absorption time for liquid added to Example 3 on the low-density side is faster than for this control, which has all of the features of Example 3 except for the gradient in the thickness direction.

These and other data suggest that the density gradient of the material of the present invention allow the material to exhibit a faster droplet absorption rate than for similar materials that do not have the density gradient in the thickness direction drawing the liquid down into the material.

In order to effectively contain the unbonded cellulosic fibers and SAP in the low-density surface of the material of the present invention during normal web handling, a layer of cellulosic tissue is hydrogen bonded to the low-density side of the sheet. By bonding this tissue to the high-density second re, effective bonding can be achieved while leaving the low-density stratum in the first region relatively uncompressed. The vertical delamination test, explained below, is a useful indicator of this bond strength. Vertical Delamination values are advantageously greater than 5N, and more desirably above 10N in order for the tissue to remain bonded during web handling and splicing typical of converting operations for disposable absorbent articles.

Vertical Delamination:

A strip of Spectape ST01 double sided adhesive tape is attached to one surface of the material to be tested. A 50 mm circular sample is cut from the taped portion using an Atom Model SE 20C die press from Associated Pacific Company of Camarillo, Calif. and an appropriately sized cutting die. A test is then performed using a Zwick Model Z005 tensile tester from Zwick/Roell in Ulm, Germany, or the equivalent. In the lower compression portion of the machine, a 50 mm diameter circular platen is attached to the load cell on the moveable crossbeam and a second larger fixed circular platen is mounted to the frame below, opposite the 50 mm moveable platen. The release paper is removed from the taped sample and it is attached to the 50 mm moveable platen using the adhesive surface. A second strip of double-sided tape is applied to the lower platen surface and the release paper is likewise removed. The platens are brought together, adhering the sample faces to both of them, and then moved apart, delaminating the sample. To do this without damaging the load cell, the moveable platen is carefully brought down onto the fixed platen with the sample between them at 30 mm/min until a force of 0.5N is read, then at 2 mm/min until a force of 5N is read, and then at 0.5 mm/min until a force of 35N is read. Then the moveable platen is moved upwards at 75 mm/min, while recording the maximum force applied as the sample delaminates. This maximum force is the vertical delamination force. Examination of the failed sample reveals whether the failure was within the sample or if the sample strength exceeded that of one of the taped bonds.

Vertical delamination testing was performed on the example materials to show the integrity of the bonds between the strata and particularly between the carrier tissue on the lower-density side of the sheet. The average of 5 values for each was recorded. Please find these in table 5 below:

TABLE 5

| | Average |
|---|---|
| | Vertical |
| | Delamination |
| | (N) |
| Example 2 | 17.3 |
| Example 3 | 17.0 |
| Example 4 | 22.9 |
| Example 5 | 22.8 |

From the forgoing, we observe the numerous modifications and variations can be effected without departing from the true spirit and scope of the novel concept of the present invention. It is to be understood that no limitation with respect to the specific embodiments illustrated here is intended or shown be inferred. The disclosure is intended to cover by the appended claims all such modifications should fall within the scope of the claims.

What is claimed:

1. An absorbent, nonwoven material exhibiting a Z-direction density gradient, comprising:
a first, relatively low-density stratum comprising a fibrous matrix of cellulosic fibrous material substantially free of synthetic fibers and binder compositions;
a second, relatively high-density stratum, juxtaposed to said first stratum in liquid-transferring relationship therewith, said second stratum comprising a fibrous matrix of cellulosic fibrous material substantially free of synthetic fibers and binder compositions,
a liquid-distribution network extending substantially through the entire thickness of said absorbent material, said liquid-distribution network being laterally adjacent to at least some portions of said first, low-density and second high-density strata in liquid-transferring relationship therewith; and
a cellulosic fiber tissue layer positioned on top of said low-density stratum, and bonded to said liquid-distribution network for integrating said absorbent material against delamination, and for inhibiting release of the fibrous material of said low-density stratum.
said material containing hydrogen bonding providing a nonwoven sheet with an MD tensile strength of at least 10N/50 mm and a vertical delamination strength of >5N, said hydrogen bonding serving to stabilize the densities of the strata and said density gradient, and stabilize the integrity of the liquid-distribution network and bonded tissue layer.

2. An absorbent, nonwoven material exhibiting a Z-direction density gradient in accordance with claim 1, wherein
said absorbent material exhibits an apparent Z-direction density gradient greater than about 1.2:1.

3. An absorbent, nonwoven material exhibiting a Z-direction density gradient in accordance with claim 1, wherein
said liquid-distribution network comprises at least one longitudinally extending densified region.

4. An absorbent, nonwoven material exhibiting a Z-direction density gradient in accordance with claim 1, wherein
said liquid-distribution network comprises a land-and-sea densified region.

5. An absorbent, nonwoven material exhibiting a Z-direction density gradient in accordance with claim 1, wherein
said liquid-distribution network comprises between about 5% and 50% of the surface area of said absorbent material.

6. An absorbent, nonwoven material exhibiting a Z-direction density gradient in accordance with claim 5, wherein
said liquid-distribution network comprises between about 10% and 35% of the surface area of said absorbent material.

7. An absorbent, nonwoven material exhibiting a Z-direction density gradient in accordance with claim 1, wherein
at least one of said strata includes superabsorbent polymeric material.

8. An absorbent, nonwoven material exhibiting a Z-direction density gradient in accordance with claim 1, wherein
said absorbent material has a basis weight of about 100-2000 gsm, and comprises between about 0% and 70%, by weight, of a superabsorbent polymeric material,
said low-density stratum having a density in the range of about 0.08 g/cc to 0.30 g/cc, and said high-density stratum having a density in the range of about 0.25 g/cc to 0.50 g/cc.

9. An absorbent, nonwoven material exhibiting a Z-direction density gradient in accordance with claim 8, wherein
said absorbent material has a basis weight of about 150-1000 gsm, and comprises between about 10% and 55%, by weight, of the superabsorbent polymeric material,
said low-density stratum having a density in the range of about 0.10 g/cc to 0.17 g/cc, and said high-density stratum having a density in the range of about 0.25 g/cc to 0.40 g/cc.

10. An absorbent, nonwoven material exhibiting a Z-direction density gradient in accordance with claim 1, wherein
said high-density stratum includes another cellulosic tissue layer at the lower surface thereof, said cellulosic tissue layers being bonded together along said liquid-distribution network.

11. An absorbent, nonwoven material exhibiting a Z-direction density gradient in accordance with claim 1, wherein
the fibrous material of said low-density stratum is different than the fibrous material of said high-density stratum.

12. An absorbent, nonwoven material exhibiting a Z-direction density gradient in accordance with claim 1, wherein the droplet absorption time ratio is >1.5:1

13. A method making an absorbent, nonwoven material exhibiting a Z-direction density gradient, comprising the steps of:
providing a cellulosic tissue layer;
depositing cellulosic material on said tissue layer;
compacting said cellulosic material to form a fibrous matrix of a first, relatively low-density stratum;
providing a second, relatively high-density stratum with hydrogen bonding formed by applying heat and pressure providing a stable density, comprising another fibrous matrix, on said first stratum; and
compacting said first and second strata by applying heat and pressure in a defined pattern, to form an absorbent material with a Z-direction density gradient, including forming a liquid-distribution network extending through the entire thickness of the material, and laterally adjacent to at least one portion of said low-density and high-density strata, wherein said cellulosic tissue layer is bonded to said liquid-distribution network for integrating said absorbent material against delamination, and for inhibiting release of the fibrous material of said first, low-density stratum.

14. A method making an absorbent, nonwoven material in accordance with claim 12, including
forming said liquid-distribution network with at least one longitudinally extending densified region.

15. A method making an absorbent, nonwoven material in accordance with claim 12, including
forming said liquid-distribution network to comprise a land-and-sea densified region.

16. A method making an absorbent, nonwoven material in accordance with claim 12, including
forming at least one of said fibrous matrices as a blend of cellulosic fibrous material and superabsorbent polymeric material.

17. A method making an absorbent, nonwoven material in accordance with claim 12, wherein
said step of providing said high-density stratum includes forming and compacting said high-density stratum separately from said low-density stratum, and thereafter positioning said high-density stratum on said low density stratum.

18. A method of making an absorbent, nonwoven material in accordance with claim 17, using an airlaid formation apparatus, comprising a single formation section, and a single bonding calender or multiple bonding calenders.
